(19) United States
(12) Patent Application Publication
(10) Pub. No.: US 2007/0005016 A1
(43) Pub. Date: Jan. 4, 2007

(54) Ocular injection device and method

(76) Inventor: David F. Williams, Minnetonka, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/156,931

(22) Filed: Jun. 20, 2005

(51) Int. Cl. A61M 5/00

(52) U.S. Cl. 604/116

(57) ABSTRACT

A device and method for an injection into an eye such as an intravitreal injection is shown. Ocular devices and methods shown provide a reduction in the number of steps involved in an injection procedure. Ocular devices and methods shown also provide consistent and repeatable location of an injection location. Other selected embodiments include integrated structures such as holding portions, or speculum structures that further facilitate an injection procedure.

### TECHNICAL FIELD

This invention relates to devices and methods to treat medical conditions of the eye. Specifically, this invention relates to devices and methods for ocular injection.

### BACKGROUND

Associated with the development of new pharmacological treatments for retinal diseases, vitreoretinal specialists are being faced with the responsibility for providing an increasing number of intravitreal injections of pharmacological agents. There is no universally accepted standard process for performing an intravitreal injection. Injections cannot always be scheduled in advance and each injection requires several steps to prepare the eye and safely perform the injection. The time required to perform injections can disrupt office schedules, resulting in unexpected prolongation of patient waiting times. A device and method to standardize and simplify the injection process is desired, thus improving patient comfort, safety and process efficiency.

What is needed is an improved ocular device and method that provides solutions to industry needs, including, but not limited to those described above.

### BRIEF DESCRIPTION OF THE DRAWINGS

FIG. 1A shows a top view of an ocular device according to an embodiment of the invention.

FIG. 1B shows a cross section view of the ocular device from FIG. 1A according to an embodiment of the invention.

FIG. 2 shows a top view of an ocular device on an eye according to an embodiment of the invention.

FIG. 3 shows a side view of an ocular device on an eye according to an embodiment of the invention.

FIG. 4 shows a flow diagram of a method according to an embodiment of the invention.

FIG. 5A shows a top view of an ocular device according to an embodiment of the invention.

FIG. 5B shows a side view of a portion of the ocular device from FIG. 5A according to an embodiment of the invention.

FIG. 6A shows a top view of an ocular device according to an embodiment of the invention.

FIG. 6B shows a side view of a portion of the ocular device from FIG. 6A according to an embodiment of the invention.

FIG. 7 shows a cross section view of an ocular device according to an embodiment of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, mechanical, chemical or logical changes, etc. may be made without departing from the scope of the present invention. Selected embodiments as described below accomplish operations such as: Anesthetize; Sterilize; Localize injection site; and Inject (ALSI) in a reduced number of steps.

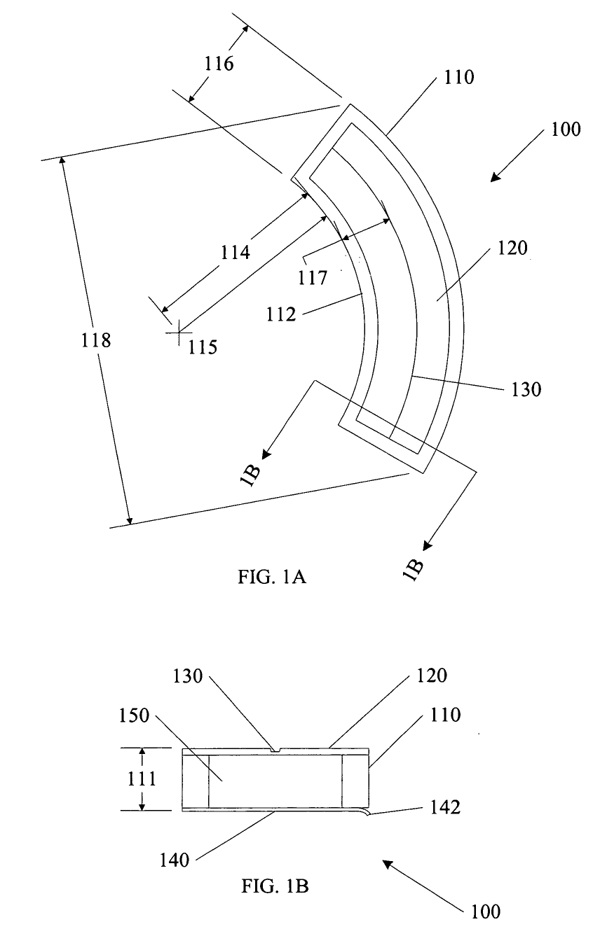

FIG. 1A shows an ocular device 100. The ocular device 100 includes a device frame 110. In one embodiment, the device frame 110 includes an alignment portion 112. In one embodiment, the alignment portion 112 is integrally formed as an edge of the device frame 110. Also shown in FIG. 1A are a membrane 120 and a target location feature 130.

In one embodiment, the device frame 110 includes a width 116 of approximately 5 mm. In one embodiment, the device frame 110 includes a length 118 of approximately 10 mm. In one embodiment, the width 116 is large enough to span a distance over a pars plana region of an eye, as will be discussed in more detail below. In one embodiment, longer lengths 118 than 10 mm are included. In one embodiment, a length is measured circumferentially along an arc, in contrast to the linear dimension shown in FIG. 1A. In one embodiment, a circumferential arc length includes arcs up to 360 degrees of a circle surrounding a cornea of an eye.

In one embodiment, the alignment portion 112 is adapted to align with a limbus of an eye, where the cornea meets the sclera, although other embodiments may align to other ocular structures. In one embodiment, the alignment portion 112 functions as a reference frame on the eye to aid in the location of other ocular features. In one embodiment where the alignment portion 112 is adapted to align with the limbus, the alignment portion 112 includes an edge with a radius 114 as determined from a center point 115. In one embodiment, the radius 114 is approximately 6 mm. This corresponds to a typical cornea diameter of 12 mm. An arcuate alignment portion 112 allows easy location in two dimensions along a surface of the eye with an identifiable feature such as the limbus. The location with the limbus in turn provides a reference frame for location of the pars plana.

In one embodiment, the target location feature 130 is spaced apart from the alignment portion 112 by a known distance. In one embodiment, the spacing between the target location feature 130 and the alignment portion 112 is a fixed distance 117. In one embodiment the location of the target location feature 130, as determined by the fixed distance 117, provides guidance to a location within the pars plana portion of the eye. In one embodiment, where the alignment portion 112 aligns with the limbus, the fixed distance is approximately 3.5 mm. In one embodiment, the spacing between the target location feature 130 and the alignment portion 112 is a variable distance that is selected by a doctor to location within the pars plana portion of the eye. In one embodiment, the target location feature 130 determines an acceptable injection area no greater than 4 mm from the limbus.

A linear target location feature 130 is shown in FIG. 1A, although the invention is not so limited. Other target location features include points, circles, other geometric areas, etc. In one embodiment, the target location feature includes a scale, such as ruler markings, that allows a doctor to select a desired distance from a reference frame such as the alignment portion 112. Although in FIG. 1A, the target location feature 130 is located directly on the membrane 120, the invention is not so limited. Other embodiments include a target location feature coupled to the device frame 110 separately without the use of a membrane.

FIG. 1B shows a cross section of the ocular device 100 from FIG. 1A. The device frame 110 is shown, with the membrane 120 coupled to a top surface of the device frame 110. In one embodiment, a thickness 111 of the ocular device 100 is approximately 3 mm. In one embodiment, the device frame 110 and other features such as the membrane 120 define a reservoir 150. Drugs or other substances contained in the reservoir will be discussed in more detail below. In one embodiment, a cover portion 140 is further included to define the reservoir 150. In one embodiment, the cover portion 140 includes a peel-off covering. FIG. 1B illustrates a lip portion 142 of the cover portion 140 that is used to peel off the cover portion in one embodiment.

The target location feature 130 is further shown in FIG. 1B. Although the target location feature 130 as shown in FIG. 1A is illustrated as a simple line, one of ordinary skill in the art, having the benefit of the present disclosure, will recognize that the target location feature 130 may have various widths or cross sections as shown in FIG. 1B. In one embodiment, the target location feature 130 includes a thinner cross section profile as shown in FIG. 1B. As will be described in more detail below, in one method of use, a needle is inserted through the membrane 120 to deliver an injection within the vitreous of a patient's eye. The thinner cross section of the target location feature 130 in one embodiment provides a lower insertion force of a needle through the membrane 120.

In one embodiment, one or more of the elements of the ocular device 100 are formed from a substantially transparent material. An example material class for the device frame 110 includes transparent polymers. In one embodiment, the device frame is formed from a resilient material such as an elastomer that allows the device frame 110 to conform to surfaces of eyes having slightly different spherical radii. In general, most eyes have a radius of 24-25 mm. In one embodiment, a resilient material for the device frame 110 includes enough flexibility to form a close interface with a surface of an eye over radii of approximately 24-25 mm. In one embodiment, a substantially transparent elastomer is used as a material for the membrane 120. In one embodiment the contents of the reservoir are substantially transparent. In use, a substantially transparent material allows a doctor to see a location on the eye beneath the ocular device 100 during the injection procedure.

In one embodiment, one or more of the elements of the ocular device 100 are formed from a compliant material. In one example, the material of a structure such as the device frame 110 is formed from a polymer material with enough mechanical integrity to be handled, but with enough compliant properties to conform to a surface of the eye during use. In one embodiment, the device frame 110 and the membrane 120 are integrally formed from a compliant material, such as an elastomer.

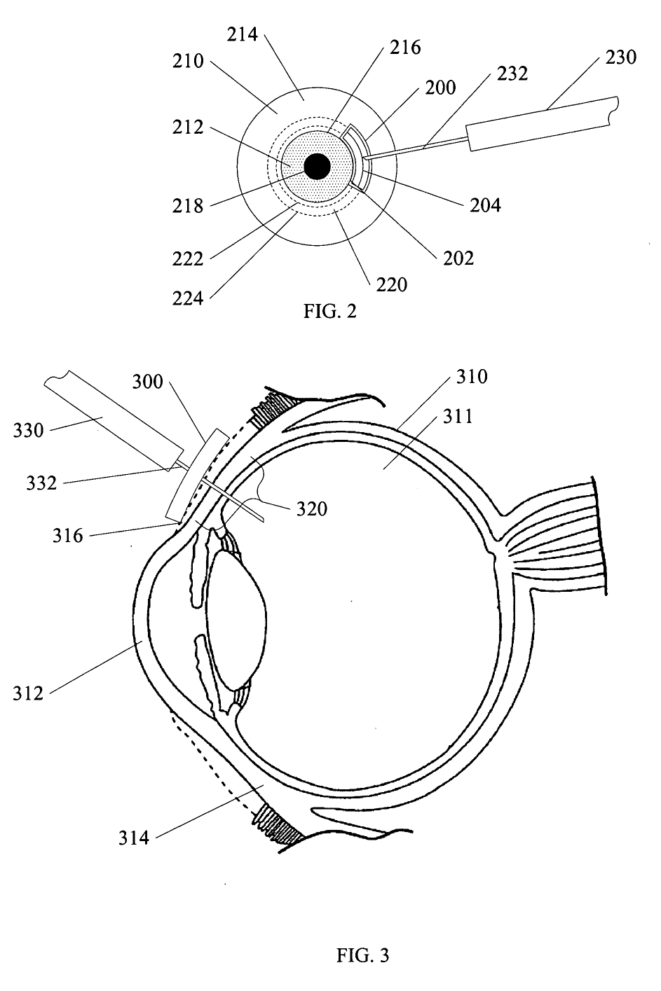

FIG. 2 shows an embodiment of an ocular device 200 similar to embodiments described above. The ocular device 200 is shown in place on an eye 210. Structures of the eye that are shown include the cornea 212 and iris 218; the sclera 214; and the limbus 216 at the intersection of the cornea 212 and the sclera 214. The pars plana region 220 is shown as a ring with an inner border 222 and an outer border 224. The pars plana region is a safe region for injections to be delivered to the vitreous of the eye.

An alignment portion 202 of the ocular device 200 is shown in alignment with the limbus 216 of the eye. When the alignment portion 202 is in place along the limbus 216, a target location feature 204 similar to embodiments described above is located over the pars plana region 220. As shown in FIG. 2, a syringe 230 or other device with a needle 232 is easily located over the target location feature 204. Advantages of the device 200 therefore include easy location and positioning over the pars plana region 220. An injection procedure is safe, repeatable and fast using ocular devices such as the device 200 and others shown in the present disclosure.

FIG. 3 shows a side view of a needle 332 of a syringe 330 similar to the one shown in FIG. 2. The needle 332 passes through the ocular device 300 into a vitreous 311 of an eye 310. The eye 310 includes a cornea 312 and a sclera 314 with a limbus 316 at the intersection as described above. Alignment of the ocular device 300 is shown along the limbus 316 with the needle 332 passing into a pars plana region 320. Any of a number of drugs are used for injections. Some examples of intravitreal injection include antibiotics, anti-angiogenic agents, anti-inflammatory agents such as steroids, etc.

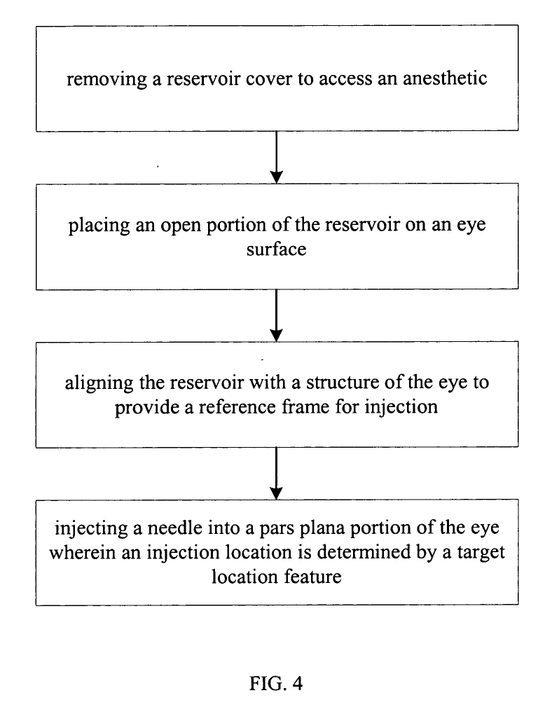

FIG. 4 shows one example of a method of use to provide an injection into an eye. In one embodiment, a reservoir cover is removed from a reservoir such as a reservoir defined in embodiments described above. In one embodiment, the reservoir includes an anesthetic. In one embodiment, the reservoir further includes a bactericide. Other substances such as inactive carrier substances are included in selected embodiments as well.

Current injection practices include application of a bactericide using a method such as eye drops, then subsequent administration of an anesthetic using drops or a local subconjunctival injection. After this process, an injection location in the pars plana region is determined and the intravitreal injection is administered.

Using an ocular device as described in the present disclosure, administration of drugs to the surface of the eye is accomplished concurrently as the intravitreal injection site is determined by the device. In one embodiment, a bactericide is applied to the surface of the eye using conventional methods, then an anesthetic is delivered from the ocular device reservoir. In one embodiment, both a bactericide and an anesthetic are mixed in the reservoir and delivered concurrently when the ocular device is positioned on the eye. Other embodiments include separate reservoirs for the bactericide and the anesthetic, as will be discussed in more detail below. In one embodiment the bactericide includes povidone iodine, although other bactericides are also within the scope of the invention. In one embodiment, 5%-10% povidone iodine solution is used. In one embodiment, the anesthetic includes lidocaine. In one embodiment the lidocaine includes lidocaine gel. In one embodiment, 2% lidocaine gel is used. An advantage of lidocaine gel includes the ability to invert the open reservoir before placing the ocular device onto the eye. The viscous nature of lidocaine gel keeps the contents of the reservoir in place while the ocular device is being positioned.

As shown in FIG. 4, in one method, the open reservoir containing one or more drugs is placed on an eye surface. The ocular device is then aligned with a structure of the eye to provide a reference frame for injection location. In one embodiment the ocular device is aligned with the limbus. Using a target location feature as described in selected embodiments above, a needle can then be inserted through a correctly determined location of the eye, for example through the pars plana region.

Embodiments of ocular devices and methods as described above provide a number of advantages. One advantage includes a reduction in the number of separate steps used in prior methods. Two or more steps such as application of a bactericide, application of an anesthetic, and determining an injection location are performed concurrently using embodiments of the present invention. Another advantage of ocular devices and methods as described above includes the consistent and repeatable location of an injection location with respect to an easily identified structure of the eye. Ocular device and methods described therefore provide reduced time to complete an injection procedure, and increased patient safety. While a number of advantages of embodiments described herein are listed above, the list is not exhaustive. Other advantages of embodiments described above will be apparent to one of ordinary skill in the art, having read the present disclosure.

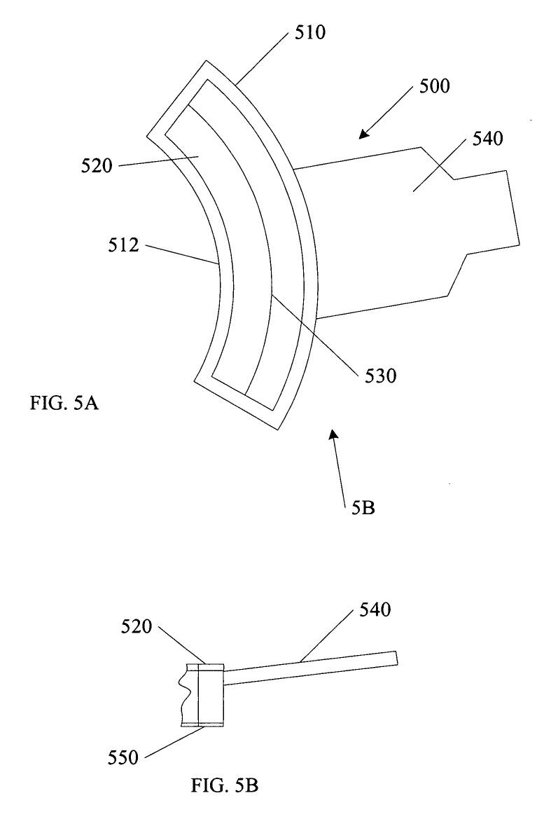

FIG. 5A shows an ocular device 500 according to another embodiment of the invention. The ocular device 500 includes a device frame 510 as shown in FIG. 5A. Similar to embodiments described above, in one example, the device frame 510 includes an alignment portion 512. In one embodiment, the alignment portion 512 is integrally formed as an edge of the device frame 510. Also shown in FIG. 5A are a membrane 520 and a target location feature 530. A holding portion 540 is shown attached to a side of the device frame 510. In one embodiment, at least one such holding portion attached at an appropriate location on the device frame 510 is used by the doctor to aid in placing and holding the ocular device 500 during a procedure.

FIG. 5B shows a side view of a portion of the ocular device 500 from FIG. 5A as viewed along line 5B. A portion of the membrane 520 is shown. In one embodiment, a peel-off strip or other cover structure 550 is included on a bottom portion of a defined reservoir. The holding portion 540 is shown from the side. In one embodiment, the holding portion 540 angles upward from the device frame 510. In one embodiment, at least one holding portion is integrally formed with the device frame 510, for example by injection molding a polymer material. Advantages of at least one holding portion include ease of placement and holding for a doctor, as well as improved field of view during an injection step. Using a holding portion, a doctor's fingers are spaced away from the target location feature 530, with which a needle is aligned.

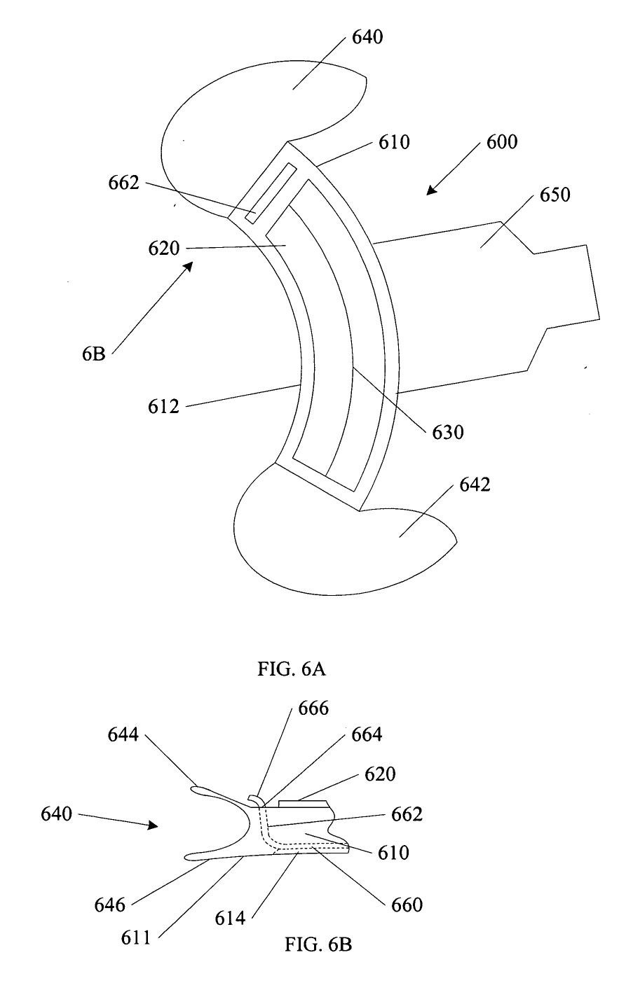

FIG. 6A shows an ocular device 600 according to another embodiment of the invention. The ocular device 600 includes a device frame 610 as shown in FIG. 6A. Similar to embodiments described above, in one example, the device frame 610 includes an alignment portion 612. In one embodiment, the alignment portion 612 is integrally formed as an edge of the device frame 610. Also shown in FIG. 6A are a membrane 620 and a target location feature 630. A first speculum portion 640 is shown attached to an end of the device frame 610, and a second speculum portion 642 is shown attached to another end of the device frame 610. In one embodiment, the first and second speculum portions 640, 642 are used to hold a patient's eyelids open during an intravitreal injection procedure. In one embodiment, similar to FIGS. 5A and 5B, a handle 650 is included to help in device placement.

In one embodiment, an opening 662 such as a slot or similar configuration is included to house a cover structure 660. One example of a cover structure 660 such as a removable strip is shown in FIG. 6B. In one embodiment, a flexible strip is used as a cover structure 660, including a vertical portion 664 and a protruding portion 666 that exits the device frame 610 at the opening 662. In one method of use, the ocular device 600 is placed on the eye and aligned as described in embodiments above. The cover portion 660 is then removed by pulling on the protruding portion 666. The cover portion 660 slides along a channel or other guide structure, and opens a reservoir within the device frame 610 similar to embodiments described above. In one embodiment, removal of the cover portion 660 allows a drug such as an anesthetic, bactericide, etc. to then flow onto a surface of the eye. An advantage of a cover portion 660 as shown in FIGS. 6A and 6B includes the ability to remove the cover portion while the ocular device 600 is in position. Material contained in the reservoir will not spill or flow out when the ocular device is inverted onto the eye.

FIG. 6B shows a side view of a portion of the ocular device 600 from FIG. 6A as viewed along line 6B. A portion of the membrane 620 is shown. The first speculum portion 640 is shown from the side. In one embodiment, at least one speculum portion is integrally formed with the device frame 610, for example by injection molding a polymer material. In one embodiment, the first speculum portion 640 includes an upper lip 644 and a lower lip 646. Although two speculum structures are shown in FIG. 6A, one continuous speculum structure, or more than two speculum structures are also within the scope of the invention. In one embodiment, for example, a continuous speculum groove is included with an arc covering an entire side of a patient's eyelids. In one embodiment two speculum structures are used for an upper and lower eyelid, while a third speculum structure is used on a side of the eye to further hold eyelids out of the working surface of the eye.

In one embodiment, the lower lip 646 follows an arc of a bottom portion 611 of the device frame 610. In one embodiment, the bottom portion 611 follows an arc that is adapted to a curvature of an eye. In one embodiment, the entire device frame 610 follows a curvature of the eye. A matched curvature at the ocular device/eye interface includes advantages such as maintaining substances within the reservoir, and keeping the substances in a local position of the eye substantially beneath the target location feature.

In one embodiment, the cover portion 660 is spaced above a bottom portion 611 of the device frame 610, thus leaving a space 614 between the bottom portion 611 and the cover portion 660. An advantage of this configuration includes the ability to remove the cover portion while in place over an eye without scraping a surface of the eye. In one embodiment, the space 614 does not contain any additional material. In one embodiment, the space 614 includes a further drug such as described in embodiments below.

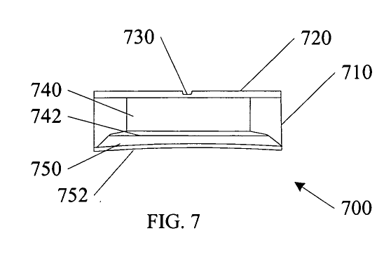

FIG. 7 shows a cross section of an ocular device 700 according to another embodiment of the invention. A device frame 710 is shown with a membrane 720 and a target location feature 730 similar to embodiments described above. In one embodiment, a first reservoir 740 is defined by structures such as the device frame 710 and the membrane 720. In one embodiment, a first cover structure 742 is included to contain the first reservoir 740. In one embodiment, the first cover structure 742 includes a peel-off strip of material.

In one embodiment, a second reservoir 750 is also included in the ocular device 700. In one embodiment, a second reservoir 750 is defined by structures such as the device frame 710 and the first cover structure 742. In one embodiment, a second cover structure 752 is included to contain the second reservoir 750. In one embodiment, the second cover structure 752 includes a peel-off strip of material.

In one embodiment, the first reservoir 740 includes an anesthetic, and the second reservoir 750 includes a bactericide. In an example method of use, the doctor would first remove the second cover structure 752 and apply the dose of bactericide to the eye, for example by dripping the bactericide, or using a swab, etc. In an example method of use, the doctor would then remove the first cover structure 742 and apply the dose of anesthetic to the eye, for example by allowing the anesthetic to flow onto the eye with the ocular device 700 inverted.

### CONCLUSION

Thus has been shown an ocular device and method to provide an injection into an eye such as an intravitreal injection. Advantages of devices and methods described above include a reduction in the number of steps involved in an injection procedure. Another advantage includes consistent and repeatable location of an injection location. Ocular device and methods described therefore provide reduced time to complete an injection procedure, and increased patient safety. Other advantages of selected embodiments include integrated structures, such as holding portions, or speculum structures that further facilitate an injection procedure.

While a number of advantages of embodiments described herein are listed above, the list is not exhaustive. Other advantages of embodiments described above will be apparent to one of ordinary skill in the art, having read the present disclosure. Although specific embodiments have been illustrated and described herein, it will be appreciated by those of ordinary skill in the art that any arrangement which is calculated to achieve the same purpose may be substituted for the specific embodiment shown. This application is intended to cover any adaptations or variations of the present invention. It is to be understood that the above description is intended to be illustrative, and not restrictive. Combinations of the above embodiments, and other embodiments will be apparent to those of skill in the art upon reviewing the above description. The scope of the invention includes any other applications in which the above structures and fabrication methods are used. The scope of the invention should be determined with reference to the appended claims, along with the full scope of equivalents to which such claims are entitled.

I claim:

1. An ocular device, comprising:
a device frame;
an alignment portion on the device frame to provide a relative location with respect to a known structure of an eye; and
a target location feature spaced apart from the alignment portion, wherein the feature provides guidance for an injection within a pars plana portion of the eye.

2. The ocular device of claim 1, further including a drug reservoir coupled to the device frame.

3. The ocular device of claim 1, wherein the target location feature is located on a membrane that allows penetration of a needle.

4. The ocular device of claim 2, wherein the drug reservoir is transparent.

5. The ocular device of claim 1, further including a dose of an anesthetic within the drug reservoir.

6. The ocular device of claim 1, further including a speculum structure coupled to the device frame.

7. The ocular device of claim 1, further including a holding portion coupled to the device frame.

8. The ocular device of claim 1, wherein an alignment portion on the device frame includes an edge shaped to align with a limbus of an eye.

9. An ocular device, comprising:
a device frame;
an alignment portion on the device frame to provide a relative location with respect to a known structure of an eye;
a target location feature spaced apart from the alignment portion, wherein the feature provides guidance for an injection within a pars plana portion of the eye; and
a reservoir coupled to the device frame containing an anesthetic.

10. The ocular device of claim 9, wherein the anesthetic includes lidocaine gel.

11. The ocular device of claim 9, wherein the reservoir contains a bactericide.

12. The ocular device of claim 11, wherein the bactericide includes povidone iodine.

13. The ocular device of claim 9, wherein the target location feature is located on a membrane that allows penetration of a needle.

14. The ocular device of claim 13, wherein the target location feature includes a line of multiple acceptable locations.

15. The ocular device of claim 14, wherein a needle penetrates locations along the line with less force than other locations on the membrane.

16. The ocular device of claim 9, further including a peel-off strip to access the reservoir.

17. The ocular device of claim 9, wherein the peel-off strip is accessible from a backside of the device frame.

18. An ocular device, comprising:
a device frame;
an alignment portion on the device frame to provide a relative location with respect to a known structure of an eye;
a target location feature spaced apart from the alignment portion, wherein the feature provides guidance for an injection within a pars plana portion of the eye;
a reservoir coupled to the device frame containing:
an anesthetic; and
a bactericide.

19. The ocular device of claim 18, further including a speculum structure integrally formed with the device frame.

20. The ocular device of claim 18, further including a holding portion integrally formed with the device frame.

21. A method, comprising:
removing a reservoir cover to access an anesthetic;
placing an open portion of the reservoir on an eye surface;
aligning the reservoir with a structure of the eye to provide a reference frame for injection; and
injecting a needle into a pars plana portion of the eye wherein an injection location is determined by a target location feature.

22. The method of claim 21, wherein removing a reservoir cover to access an anesthetic includes removing a reservoir cover to access a combined anesthetic and bactericide in the reservoir.

23. The method of claim 21, further including accessing a separate bactericide reservoir and application of bactericide.

24. The method of claim 21, wherein aligning the reservoir with a structure of the eye includes aligning the reservoir with the limbus.

25. The method of claim 21, wherein injecting a needle into the pars plana portion of the eye includes injecting through a membrane and through the reservoir into the pars plana portion of the eye.

26. The method of claim 21, further including holding eyelids open using a speculum structure coupled to the reservoir.
